(19) United States
(12) Patent Application Publication
(10) Pub. No.: US 2008/0241321 A1
(43) Pub. Date: Oct. 2, 2008

(54) Method and Device For Sorting and Packaging of Foods

(76) Inventor: Sverre Stenbom, Vastra Frolunda (SE)

Correspondence Address: HARNESS, DICKEY & PIERCE, P.L.C., P.O. BOX 8910, RESTON, VA, 20195, US

(21) Appl. No.: 11/579,346

(22) PCT Filed: May 4, 2005
(86) PCT No.: PCT/SE2005/00657

§ 371 Date: Nov. 2, 2006

(30) Foreign Application Priority Data
May 4, 2004 (SE) 0401162.3

(51) Int. Cl. G01N 33/02; G06F 7/00

(52) U.S. Cl. 426/232; 700/213

(57) ABSTRACT

A method for packaging of food products of varying sizes, in which at least two food products are to be packages in the same packing, is disclosed. The method includes making an estimate of the quantity of each food product, registering the location of each food product on an object conveyor, moving each food product to a collection area, determining by way of a computer program whether a food product is to be moved to a portion carrier and, if such move is determined, also determining to which portion carrier the food product is to be moved, raising the food product to be moved to a portion carrier, and moving the food product to the selected portion carrier and releasing the food product to the portion carrier.

### FIELD OF THE INVENTION

The present invention relates to a method for packaging of food products of varying sizes, in which at least two food products are to be packaged in the same packing. The invention also relates to a device for use in the method.

### BACKGROUND ART

When packaging soft food products, such as fresh chicken, fish and other meat products, it is important to reduce handling to a minimum. Packaging of such products usually occurs manually, i.e. they are packaged manually by sorters standing along a conveyor. To simplify the handling of the packages, it is today frequently required that all packages have the same weight. For instance, it is easier for a food shop to keep the stock in good order if all packages of chicken breasts have the same weight. In traditional manual packaging it is usually difficult to succeed in getting the exact weight or a weight with an error of just a few percent. The weight stated on a package is not allowed to be lower than a minimum by a few grammes, but there is no maximum weight, which results in great losses for the manufacturer of the packaged product. Each product can also be weighed so as to reduce these losses, but it is difficult for the sorter to manage to get weighing done and make an estimate to determine in which packing a product is to be placed in order to optimise the weight, or alternatively cut a product to achieve the appropriate weight. In cutting, at least one cut product is thus obtained. As a rule, the consumers want to obtain “whole” products.

### SUMMARY OF THE INVENTION

The object of the present invention is to suggest a solution to the above-discussed problems by providing an improved method for packaging food products, and a portioning device.

According to the invention, this object is achieved by a method according to claims 1-5. The object is also achieved by a device according to claims 6-11.

The inventive method for packaging of food products of varying sizes, in which at least two food products are to be packaged in the same packing, comprises the steps of making an estimate of the quantity of each food product, determining by means of a computer program whether a food product is to be moved to a portion carrier and, if such move is determined, also determining to which portion carrier the food product is to be moved, raising the food product to be moved to a portion carrier, and moving the food product to the selected portion carrier and releasing the food product to the portion carrier. Thus, the device comprises an object conveyor on which said food products are conveyed, a collecting means adjacent to a moving means which allows motion of the collecting means horizontally and vertically, a plurality of portion carriers, quantity estimating means, calculating means and a control device, the quantity estimating means being arranged to estimate the quantity of a food product on the object conveyor, which means is further connected to the calculating means so that information is transferable from the quantity estimating means to the calculating means, said calculating means comprising a computer program for calculating to which of said plurality of portion carriers a food product is to be moved to achieve a preset total quantity of food products for a portion carrier, the calculating means being connected to the control device, said control device being arranged to control the work of the collecting means. The present invention is thus used to package food products by first conveying them on an object conveyor past a quantity estimating means which either directly or indirectly supplies data to be able to calculate the weight of the food product. Also the location on the object conveyor is registered. The object conveyor then moves the food products on to a collection area where the food products can be picked up by a collecting means so as to be moved to a portion carrier by a moving means. A calculating means receives information from the quantity estimating means and at the same time keeps track of the thus achieved total weight of at least two portion carriers and a desired final weight to be able to determine in which portion carrier, if any, a food product is to be placed to come as close as possible to a desired final weight. The more portion carriers that are available to receive the food products, the less waste arises.

In the first place, the invention concerns soft food products. By soft food products are meant all types of foods which are not frozen and which have a natural distribution in terms of shape and weight. Moreover, quantity relates to weight, volume, length etc. but not the number of food products.

In one embodiment of the invention, the quantity is estimated by a balance which directly can supply weight data to the calculating means. In an alternative embodiment, the size is read by a size scanner as the food products pass by on the object conveyor. In the alternative embodiment, the volume of the food product is then calculated, which volume then, together with a density programmed in the calculating means, gives the weight of the food product.

In one embodiment of the invention, the collecting means is a gripping means, i.e. a means which grips the food product in a manner similar to a human hand lifting an object.

In an alternative embodiment, the collecting means raises the food products by negative pressure. The collecting means then comprises a volume with a connection to a pump and with an opening which is arranged to be moved towards a food product and subsequently, by negative pressure in the volume, hold the food product.

### BRIEF DESCRIPTION OF FIGURES

The invention will be described in more detail in the following with reference to the accompanying schematic Figures which for the purpose of exemplification illustrate embodiments of the invention.

FIG. 1 is a perspective view of an embodiment of a portioning device according to the present invention.

FIG. 2 is a perspective view of an alternative embodiment of a portioning device according to the present invention.

### DESCRIPTION OF PREFERRED EMBODIMENTS

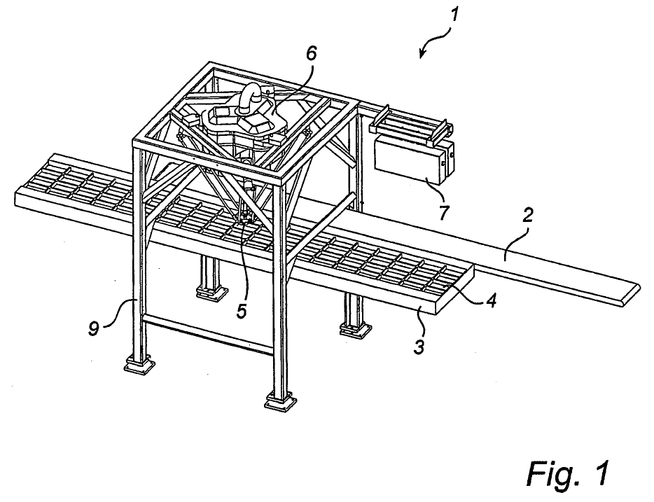

The portioning device 1 in FIG. 1 comprises an object conveyor 2, a conveyor 3 for portion carriers 4, a collecting means 5 and a moving means 6. The device 1 also comprises a size scanner 7. The moving means 6 is supported by a stand 9. In use, the food products (not shown) that are to be packaged are conveyed to the device 1 on the object conveyor 2. The food products pass under a size scanner 7 which reads the size of the food products, and a program uses the size data to calculate the volume of the product, by which volume the weight can then be calculated based on an assumed/preset density. The measuring operation is preferably performed by a signal being sent to the calculating system as a food product passes. The calculating system has a pulse counter connected to the object conveyor 2, and the length of the product is calculated as the product of the time between the beginning and the end of the product and the speed which is given by the number of pulses per unit of time. The program then knows the calculated weight and the location on the object conveyor 2. Measured values are sent to the calculating means via a network or serial communication. Depending on the food product, sorting can be done, for instance, according to weight or length or a combination of weight and length.

Another conveyor 3 carrying portion carriers 4 runs parallel to the object conveyor 2. Product carriers can be, for example, trays on which food products are to be packaged. In the embodiment shown, the portion carriers 4 are a plurality of compartments over which a plastic film is arranged. The food products are placed on the plastic film and the various portions are separated from each other by walls. When the portion carriers 4 have been filled with the requested quantity of foods, another film is placed over the food products and welded to the subjacent film. After that, the various portion packages can be separated, for example, by cutting open the weld line.

The computer program is preset to package a certain quantity in each portion carrier, and as the food products enter the working area of the moving means 6, the calculating means controls by means of the computer program the moving means 6 in order to raise by the collecting means 5 the food products that are selected to portion carriers 4. In the device 1 illustrated, the number of portion carriers 4 among which the computer can choose are 16-20 (four across the conveyor 3 and four or five along the conveyor 3), which results in very high accuracy. The food products that possibly cannot be used fall down in a collector (not shown) and/or are conveyed to another packaging unit. The smaller number of portion carriers 4 among which the computer can choose, the more products pass by the device 1. This can be used if, for instance, a plurality of devices 1 are arranged in series. Other products that pass are the food products which are outside preset tolerances, such as those being too heavy, too short etc, which continue to the end of the conveyor and are there handled as desired. From the measuring operation, the calculating means receives input data for a calculation in order to combine incoming products to portions with a preset weight. A portion thus consists of two or more products that are collected in a portion carrier 4.

With the technique that is available, i.e. when writing this text, a device according to the present invention can move about 120 products in a minute. This can be compared with a normal packaging station with eight sorters who manage 350 products in a minute. In other words, three devices according to the present invention can package more than can eight sorters, while at the same time the accuracy in terms of weight per package increases, thus reducing the losses.

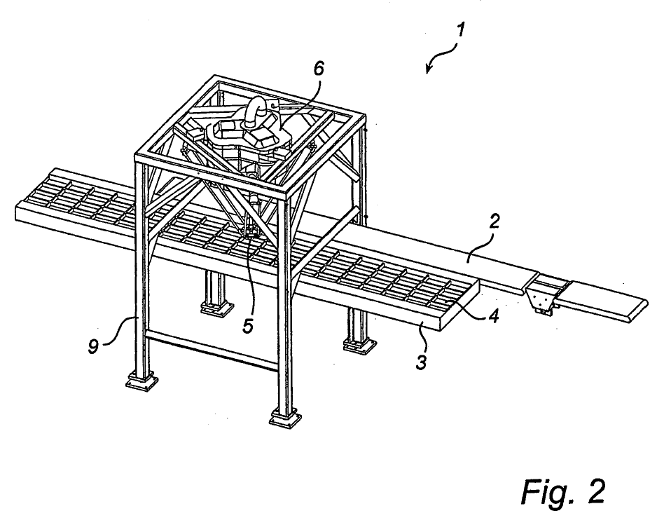

FIG. 2 shows an alternative embodiment of the device 1 according to the invention. A balance 8 is here instead used as the quantity estimating means. The accuracy in using a balance 8 is greater to estimate the weight, which also results in increased accuracy of the final weight of a package with food products. A drawback, however, is that instead of one conveyor three conveyors will be necessary, one for moving the products up to the balance, one for the actual balance and a third for moving the products from the balance and on. When the device is adapted to handle soft food products, which may sometimes be slightly sticky, residues of products can get stuck in the borders between the conveyors, thus causing problems, for instance in cleaning.

It will be appreciated that many modifications of the above-described embodiments of the invention are possible within the scope of the invention as defined by the appended claims. For example, not all portion carriers 4 along the width of the conveyor 3 need be intended for the same final weight. Of four portion carriers side by side, for instance two may be intended for 1000 g and the other two for 500 g. It is also conceivable to determine other options for sorting, which are then adjusted to product requirements so that, for instance, the products are to be sorted so that the difference between a large and a small product in the same packing is within a pre-set limit. The motion of the object conveyor 2 and the other conveyor 3 can also be different. However, the motion of the object conveyor 2 suitably is continuous while the other conveyor 3 moves in steps so that it is moved, for example, the length of a portion carrier 4 in each step.

Of course, it is also possible to use the invention for foods that are not soft.

1. A method for packaging of food products of varying sizes, in which at least two food products are to be packaged in the same packing, comprising:
making an estimate of the quantity of each food product;
registering the location of each food product on an object conveyors;
moving each food product further on the object conveyor to a collection area;
determining, via a computer program, whether a food product is to be moved to one portion carrier out of at least two portion carriers across a portion carrier conveyor, and at least four along the conveyor, and if such move is determined, also determining to which portion carrier the food product is to be moved;
raising the food product to be moved to a portion carrier; and
moving the food product to the selected portion carrier and releasing the food product to the portion carrier.

2. A method as claimed in claim 1, wherein said food products are soft.

3. A method as claimed in claim 1, wherein said estimate of quantity is performed by reading the size and calculating the volume of the food product.

4. A method as claimed in claim 3, wherein the weight of the food product is calculated by at least one of an estimated and a preset density of the food product.

5. A method as claimed in claim 1, wherein said estimate of quantity is performed by weighing the food product.

6. A portioning device, comprising:
an object conveyor on which food products are conveyed;
a collecting device, adjacent to a moving device which allows motion of the collecting device horizontally and vertically;
a plurality of portion carriers arranged such that there are at least two portion carriers across the portion carrier conveyor and at least four along the conveyor;
a quantity estimating device;
a calculating device; and
a control device, the quantity estimating device being arranged to estimate the quantity of a food product on the object conveyor, the quantity estimating device being further connected to the calculating device so that information is transferable from the quantity estimating device to the calculating device, the calculating device including a computer program for calculating to which of said portion carriers a food product is to be moved to achieve a preset total quantity of food products for a portion carrier, the calculating device being connected to the control device, said control device being arranged to control the work of the collecting device, wherein the object conveyor is arranged to move the food products from the quantity estimating device to the working area of the collecting device.

7. A portioning device as claimed in claim 6, wherein said quantity estimating device is a balance.

8. A portioning device as claimed in claim 6, wherein said quantity estimating device is a size scanner.

9. A portioning device as claimed in claim 6, wherein said collecting device is a gripping device.

10. A portioning device as claimed in claim 6, wherein said collecting device includes a volume with a connection to a pump and with an opening, which is arranged to be moved towards a food product and subsequently, by negative pressure in the volume, hold the food product.
